(19) United States
(12) Patent Application Publication
(10) Pub. No.: US 2014/0107463 A1
(43) Pub. Date: Apr. 17, 2014

(54) RADIATION IMAGE CAPTURING SYSTEM AND RADIATION IMAGE CAPTURING METHOD

(71) Applicant: FUJIFILM Corporation, Tokyo (JP)

(72) Inventors: Naoyuki NISHINO, Ashigarakami-gun (JP); Haruyasu NAKATSUGAWA, Ashigarakami-gun (JP); Yasunori OHTA, Ashigarakami-gun (JP); Naoto IWAKIRI, Ashigarakami-gun (JP)

(73) Assignee: FUJIFILM Corporation, Tokyo (JP)

(21) Appl. No.: 14/134,857

(22) Filed: Dec. 19, 2013

Related U.S. Application Data
(63) Continuation of application No. PCT/JP2012/066531, filed on Jun. 28, 2012.

(30) Foreign Application Priority Data
Jun. 30, 2011 (JP) 2011-146656

(51) Int. Cl. A61B 6/00

(52) U.S. Cl. CPC A61B 6/4208
USPC 600/407

(57) ABSTRACT

A radiation image capturing system and a radiation image capturing method, wherein this radiation image capturing system comprises a radiation source, a radiation detection device provided with a radiation detector for converting radiation that has been transmitted through at least a subject from the radiation source into radiation image information, a photographing sequence setting unit for setting the photographing sequence of radiation photographing when successive radiation photographing is performed, and a photographing sequence display unit (a display device, a cassette display unit, a terminal display unit) for displaying the set photographing sequence.

### CROSS-REFERENCE TO RELATED APPLICATIONS AND PRIORITY CLAIM

This application is a Continuation of International Application No. PCT/JP2012/066531 filed on Jun. 28, 2012, which was published under PCT Article 21(2) in Japanese, which is based upon and claims the benefit of priority from Japanese Patent Application No. 2011-146656 filed on Jun. 30, 2011, the contents all of which are incorporated herein by reference.

### TECHNICAL FIELD

The present invention relates to a radiographic image capturing system including a radiation detecting device for converting radiation from a radiation source that has passed through a subject into radiographic image information, and a radiographic image capturing method using the radiographic image capturing system.

### BACKGROUND ART

In the medical field, portable radiographic image capturing apparatus such as an FPD (Flat Panel Detector) have been used for detecting the intensity of radiation that has passed through a human body in order to capture an image of the inside of the human body. An FPD (hereinafter referred to as an “electronic cassette”) can be used flexibly on patients who cannot move, because the electronic cassette is capable of capturing images of a patient lying on a bed or the like, and can be changed in position in order to adjust the areas to be imaged.

Electronic cassettes include an indirect-conversion-type electronic cassette having a scintillator for temporarily converting radiation into visible light, and a solid-state detector for converting visible light into electric signals. In particular, an electronic cassette including a scintillator made of CsI (cesium iodide) has a high response speed and a high detection capability, and hence is of high performance.

However, an electronic cassette having a scintillator made of CsI tends to suffer from a so-called bright-burn phenomenon, which is a type of afterimage, as a phenomenon unique to CsI scintillators. Bright-burn phenomena occur especially if the scintillator is irradiated with intense radiation. According to a radiographic image capturing process, the electronic cassette captures an image with radiation at an increased dose, and thereafter, an image is captured again with radiation. If the electronic cassette captures an image with radiation at an increased dose, many traps are developed unevenly in the scintillator. If an image is captured again with radiation using the electronic cassette, information represented by the traps is added as radiographic image information and is output from the scintillator. The scintillator tends to bring about irregular sensitivity rises due to bright-burn phenomena, which result in a reduction in contrast and hence a drop in image quality. These problems lead to a reduction in accuracy if subjects are diagnosed by interpreting the captured image.

Heretofore, methods have been proposed for minimizing bright-burn phenomena, as disclosed in Japanese Laid-Open Patent Publication No. 2003-107163, Japanese Laid-Open Patent Publication No. 2010-523997 (PCT), and Japanese Laid-Open Patent Publication No. 2009-514636 (PCT).

According to Japanese Laid-Open Patent Publication No. 2003-107163, the scintillator is heated to discharge electric charges held by deep traps.

According to Japanese Laid-Open Patent Publication No. 2010-523997 (PCT), after a radiographic image has been captured, ultraviolet radiation is applied to the scintillator from a side opposite to an X-ray-irradiated surface thereof, thereby causing the scintillator to emit light, and image information generated by the emitted light is used to perform a correction (calibration).

According to Japanese Laid-Open Patent Publication No. 2009-514636 (PCT), a main image capturing process is preceded by application of radiation to the scintillator in order to form deep traps in the scintillator over the entirety thereof, thereby holding local sensitivity rises to a minimum.

The bright-burn phenomenon is generally referred to as an afterimage phenomenon. Afterimage phenomena also occur in direct-conversion-type electronic cassettes made of selenium, and are referred to as “ghost” phenomena. Similar to the case of bright-burn phenomena, ghost phenomena occur due to electric charges, which remain in selenium in from a preceding image capturing process, and are added and output as radiographic image information in a subsequent image capturing process. Thus, the scintillator tends to bring about irregular sensitivity rises due to such ghost phenomena, which leads to a reduction in contrast and hence a drop in image quality.

Heretofore, an attempt has been made to reduce the occurrence of ghost phenomena with an upper electrode, which is disposed directly in physical and electric contact with an electric charge generator layer that includes a base made of amorphous selenium (see Japanese Laid-Open Patent Publication No. 2006-263452). According to another prior art example, an upper electrode is disposed over an electric charge generator layer, which includes a base made of amorphous selenium with a non-insulating organic layer interposed therebetween, thus making it possible to transport electric charges across the non-insulating organic layer in order to reduce the occurrence of ghost phenomena (see Japanese Laid-Open Patent Publication No. 2007-199065 and Japanese Laid-Open Patent Publication No. 2007-296337). Since there is no electric charge barrier layer, thin-film transistors, which are coupled with signal storage capacitors, are likely to experience break down upon exposure to intensive radiation. However, a structure for positively passing a leakage current is employed in order to prevent the thin-film transistors from breaking down.

### SUMMARY OF INVENTION

The method disclosed in Japanese Laid-Open Patent Publication No. 2003-107163 is problematic in that, since the scintillator needs to be heated, a certain period of time is required after an image is captured until the scintillator can be heated to discharge electric charges held by the deep traps. Hence, the disclosed method is not applicable to an image capturing process for capturing successive images in a short period of time.

The method disclosed in Japanese Laid-Open Patent Publication No. 2010-523997 (PCT), which obtains a corrective image in advance by applying ultraviolet radiation to the scintillator from a side opposite to the X-ray-irradiated surface, may not necessarily be capable of generating an accurate corrective image, since the amount of light emitted from the scintillator upon exposure to ultraviolet radiation is small. Another problem is that, inasmuch as the casing and internal structural members of the electronic cassette must be made of a material that is permeable to ultraviolet radiation, the degree of freedom in designing the electronic cassette is low, which poses a limitation on efforts to reduce the cost of the electronic cassette.

The method disclosed in Japanese Laid-Open Patent Publication No. 2009-514636 (PCT) is disadvantageous in that, since bright-burn is a phenomenon that lasts a few days, it is doubtful that the electronic cassette can be controlled after bright-burning thereof has been caused intentionally.

The approaches disclosed in Japanese Laid-Open Patent Publication No. 2006-263452, Japanese Laid-Open Patent Publication No. 2007-199065, and Japanese Laid-Open Patent Publication No. 2007-296337 are problematic in that, since the upper electrode must be made of a material that has a lower work function than the electric charge generator layer disposed beneath the upper electrode, and which is chemically stable if placed in contact with selenium, the electronic cassette is designed with a low degree of freedom, which poses a limitation on efforts to reduce the cost of the electronic cassette. In addition, the operation timings and circuit arrangements thereof need to be reconfigured in order to prevent a leakage current, which is passed positively upon exposure to strong radiation, from adversely affecting gate drivers and output circuits. Consequently, the circuit arrangements are likely to be complicated and highly costly.

The present invention has been made in view of the aforementioned drawbacks. It is an object of the present invention to provide a radiographic image capturing system and a radiographic image capturing method, which are capable of capturing radiographic images while staying clear of regions where afterimage phenomena (bright-burn or ghost phenomena) tend to occur in a series of radiographic image capturing processes, thereby preventing the S/N ratio and contrast from being lowered, and which are reduced in cost without lowering the degree of freedom in designing the radiation detecting device.

[1] A radiographic image capturing system according to a first aspect of the invention comprises a radiation source, a radiation detecting device including a casing and a radiation detector housed in the casing for converting radiation emitted from the radiation source and that has passed through at least a subject into radiographic image information, an image capturing sequence setting unit for setting a radiographic image capturing sequence for carrying out a series of radiographic image capturing processes, and an image capturing sequence display unit for displaying the set radiographic image capturing sequence. The series of radiographic image capturing processes represents radiographic image capturing processes that range from frontal to lateral radiographic image capturing processes or from lateral to frontal radiographic image capturing processes, and basically, refers to two or more radiographic image capturing processes according to a radiographic image capturing sequence designated by one image capturing order. The series of radiographic image capturing processes does not include radiographic image capturing processes that cover two persons.

[2] In the first aspect of the invention, the image capturing sequence setting unit may include a projected area estimator for estimating a projected area of an image capturing region, which is projected onto the radiation detecting device prior to the series of radiographic image capturing processes, and the image capturing sequence setting unit may set the radiographic image capturing sequence such that the projected area will change from a greater value to a smaller value during the radiographic image capturing processes.

[3] In the first aspect of the invention, the image capturing sequence setting unit may set the radiographic image capturing sequence such that at least one of a tube voltage, a tube current, and time will change from a smaller value to a greater value during the radiographic image capturing processes.

[4] In the first aspect of the invention, the image capturing sequence setting unit may set the radiographic image capturing sequence such that a tube voltage will change from a smaller value to a greater value during the radiographic image capturing processes.

[5] In the first aspect of the invention, the image capturing sequence display unit may be connected to a controller, which controls at least the radiation source and the radiation detecting device.

[6] In the first aspect of the invention, the image capturing sequence display unit may be mounted on the casing of the radiation detecting device.

[7] In the first aspect of the invention, the image capturing sequence display unit may be mounted on a portable information terminal, which is carried by an operator of the radiographic image capturing system.

[8] In the first aspect of the invention, the radiation detecting device may comprise a scintillator using CsI (cesium iodide) for temporarily converting the radiation into visible light, and solid-state detecting elements for converting the visible light into electric signals.

[9] A radiographic image capturing method according to a second aspect of the invention uses a radiation source and a radiation detecting device including a casing and a radiation detector housed in the casing for converting radiation emitted from the radiation source and that has passed through at least a subject into radiographic image information. The radiographic image capturing method comprises an image capturing sequence setting step of setting a radiographic image capturing sequence for carrying out a series of radiographic image capturing processes, and an image capturing sequence display step of displaying the set radiographic image capturing sequence.

[10] In the second aspect of the invention, the image capturing sequence setting step may further comprise a projected area estimating step of estimating a projected area of an image capturing region, which is projected onto the radiation detecting device prior to the series of radiographic image capturing processes, and the image capturing sequence setting step may set the radiographic image capturing sequence such that the projected area will change from a greater value to a smaller value during the radiographic image capturing processes.

[11] In the second aspect of the invention, the image capturing sequence setting step may set the radiographic image capturing sequence such that at least one of a tube voltage, a tube current, and time will change from a smaller value to a greater value during the radiographic image capturing processes.

[12] In the second aspect of the invention, the radiation detecting device may comprise a scintillator using CsI (cesium iodide) for temporarily converting the radiation into visible light, and solid-state detecting elements for converting the visible light into electric signals.

With the radiographic image capturing system according to the present invention, before a series of radiographic image capturing processes are carried out, it is possible to set a radiographic image capturing sequence so as to capture radiographic images that stay clear of regions where afterimage phenomena (bright-burn or ghost phenomena) tend to occur, and to display the set radiographic image capturing sequence. By performing radiographic image capturing processes according to the displayed radiographic image capturing sequence, the operator can capture radiographic images in which regions where afterimage phenomena occur are excluded. As a result, the S/N ratio and contrast are prevented from being lowered, and a reduction in cost can be achieved without lowering the degree of freedom in designing the radiation detecting device.

### BRIEF DESCRIPTION OF DRAWINGS

FIG. 1 is a diagram of a radiographic image capturing system according to an embodiment of the present invention;

FIG. 2 is a vertical cross-sectional view of a radiation detecting device;

FIG. 3 is a circuit diagram of a circuit arrangement of the radiation detecting device;

FIG. 4 is a block diagram of the radiation detecting device;

FIG. 5 is a block diagram showing exchange of signals between a console, a portable information terminal, the radiation detecting device, and a display device, with respect to a sequence of image capturing processes;

FIG. 6A is a diagram showing a radiographic image capturing process for capturing an image of a side of a chest, together with an area of the irradiated surface of the radiation detecting device that is irradiated with radiation;

FIG. 6B is a diagram showing a radiographic image capturing process for capturing an image of a frontal region of a chest, together with an area of the irradiated surface of the radiation detecting device that is irradiated with radiation;

FIGS. 7A and 7B are diagrams showing examples of messages;

FIGS. 7C and 7D are diagrams showing examples of illustrations; and

FIG. 8 is a flowchart of a processing sequence of the radiographic image capturing system.

### DESCRIPTION OF EMBODIMENTS

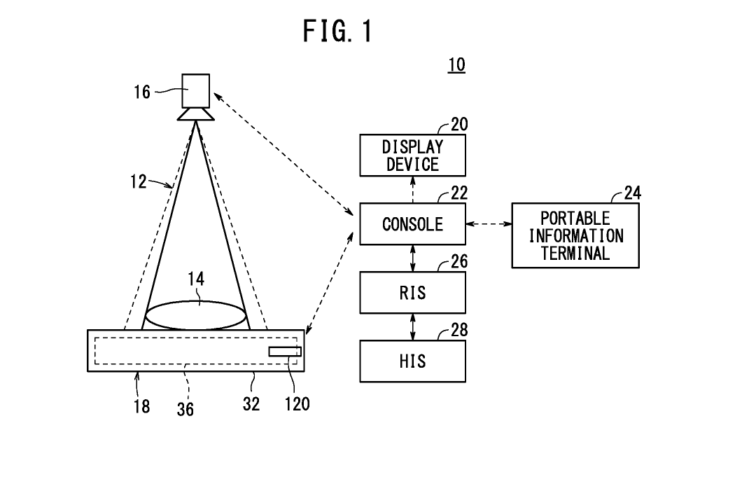

A radiographic image capturing system according to an embodiment of the present invention will be described below with reference to FIGS. 1 through 8.

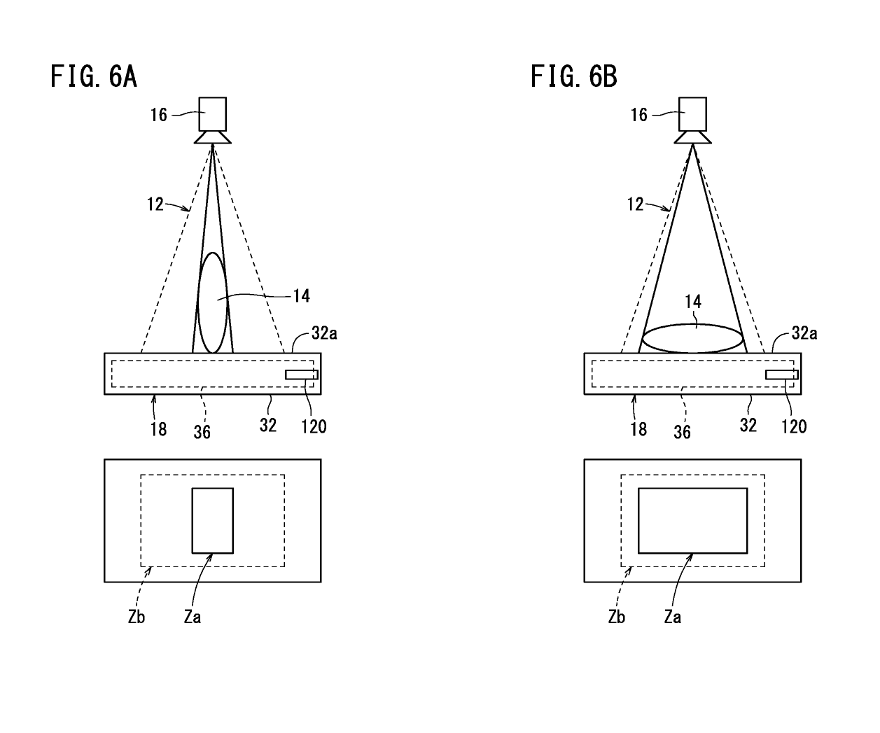

As shown in FIG. 1, a radiographic image capturing system 10 according to the present embodiment includes a radiation source 16 for applying radiation 12 at a dose according to image capturing conditions to a subject (such as a patient or the like) 14, a radiation detecting device 18 for detecting radiation 12 that has passed through the subject 14, a display device 20 for displaying a radiographic image based on radiation 12 detected by the radiation detecting device 18, a console 22 (controller) for controlling the radiation source 16, the radiation detecting device 18, and the display device 20, and an image capturing switch for the radiation source 16. The radiographic image capturing system 10 also includes a portable information terminal 24, which is carried by an operator, for confirming states including image capturing processes. Signals are sent and received between the console 22, the radiation source 16, the radiation detecting device 18, the portable information terminal 24, and the display device 20 through wireless communication links, for example. The console 22 is connected to a radiology information system (RIS) 26, which generally manages radiographic image information handled by a radiological department of a hospital in which the radiographic image capturing system 10 is installed, along with managing other additional information. The RIS 26 is connected to a hospital information system (HIS) 28, which generally manages medical information in the hospital. In FIGS. 1, 6A, and 6B, furthermore, radiation 12 that passes through the subject 14 is indicated by the solid lines, and radiation 12 that does not pass through the subject 14 is indicated by the dotted lines.

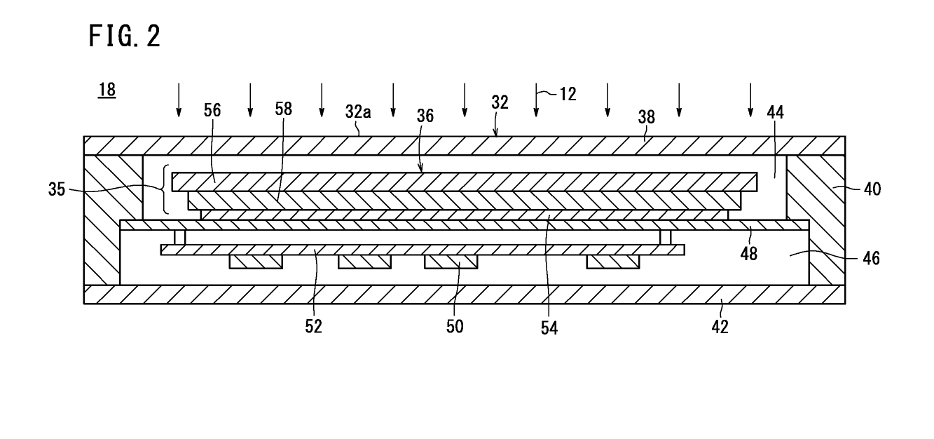

As shown in FIG. 2, the radiation detecting device 18 includes a casing 32 made of a material permeable to radiation 12 from the radiation source 16, and a radiation detector 36 having a converter 35 for converting radiation 12 from the radiation source 16 that has passed through at least the subject 14.

The casing 32 has a substantially flat front plate 38 providing a front surface (irradiation surface 32a) that is irradiated with radiation 12, a frame 40 providing side surfaces, a substantially flat rear plate 42 providing a rear surface, and a substantially flat partition 48 disposed in the frame 40, which divides the housing space in the casing 32 into a first compartment 44 near the front plate 38 and a second compartment 46 near the rear plate 42. At least one circuit board 52 with various electronic components 50 mounted thereon is disposed on a rear surface of the partition 48.

The radiation detector 36 is disposed in the first compartment 44, which is surrounded by the front plate 38, the frame 40, and the partition 48. The radiation detector 36 is fixed to the partition 48 by a support plate 54.

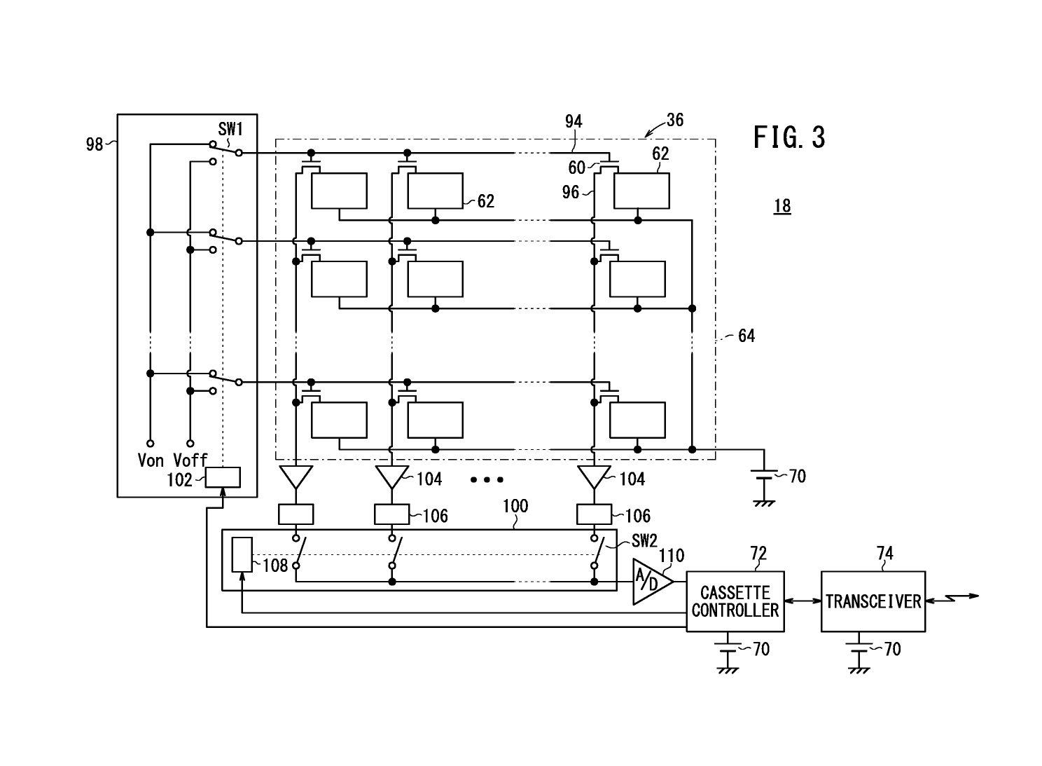

The converter 35 of the radiation detector 36 is a surface reading type, i.e., an ISS (Irradiation Side Sampling) type of converter, including a photoelectric transducer board 56 providing a front surface of the converter 35, and a scintillator 58 providing a rear surface of the converter 35. The scintillator 58 is made of a phosphor including a base material of GOS (Gd₂O₂S:Tb) CsI:Tl, or the like for converting radiation 12 that has passed through the subject 14 into visible light. The photoelectric transducer board 56 comprises an array of thin-film transistors (TFTs) 60 (see FIG. 3), and a photoelectric conversion layer 64 having solid-state detecting elements 62 (see FIG. 3, hereinafter also referred to as “pixels 62”) made of a material such as amorphous silicon (a-Si) for converting visible light into electric signals, the photoelectric conversion layer 64 being disposed on the array of TFTs 60. In other words, the converter 35 comprises the scintillator 58, which functions as a radiation to visible light converter, and the photoelectric conversion layer 64, which functions as a visible light to electric signal converter.

Since, in the ISS-type converter 35, radiation 12 passes through the photoelectric transducer board 56 to the scintillator 58, the photoelectric transducer board 56 must prevent absorption of radiation 12 as much as possible.

The photoelectric transducer board 56 is constructed of an insulating substrate, not shown, the TFTs 60, and the photoelectric conversion layer 64, which are stacked successively along a direction in which radiation 12 is applied. The photoelectric conversion layer 64, which is positioned near the scintillator 58, absorbs electromagnetic waves, e.g., visible light, emitted from the scintillator 58, and generates electric charges depending on the absorbed visible light. More specifically, the photoelectric conversion layer 64 preferably includes a photoelectric conversion film made of a-Si, or an organic photoconductor (OPC) material, or the like. The TFTs 60, which read electric charges generated by the photoelectric conversion layer 64, preferably include an active layer of a-Si, an amorphous oxide, an organic semiconductor material, carbon nanotubes, or the like. The photoelectric transducer board 56, which includes the aforementioned materials, can be fabricated according to a low-temperature process, so as to be flexible and minimize absorption of radiation 12.

The scintillator 58 is fabricated by forming columnar crystals of CsI along a direction in which radiation 12 is applied on an evaporated substrate, not shown, disposed on the surface of the photoelectric transducer board 56, which faces toward the rear surface of the casing 32. If the scintillator 58 is made of columnar crystals of thallium-added cesium iodide (CsI:Tl), and the photoelectric conversion layer 64 is made of quinacridone as an OPC, then the difference between the peak wavelength of light emitted by the scintillator 58 and the peak wavelength of light absorbed by the photoelectric conversion film can be reduced to 5 nm or smaller, for thereby maximizing the amount of electric charges generated by the photoelectric conversion layer 64. The evaporated substrate may comprise a thin aluminum (Al) substrate, which is highly resistant to heat and low in cost.

The material of the scintillator 58 is not limited to CsI or CsI:Tl, but may be CsI:Na (sodium-activated cesium iodide), GOS (gadolinium oxide sulfur, Gd₂O₂S:Tb), or the like. According to the present embodiment, the converter 35 may be of the reverse side reading type, i.e., a PSS (Penetration Side Sampling) type, in which the scintillator 58 and the photoelectric transducer board 56 are disposed successively along the direction in which radiation 12 is applied. Alternatively, the converter 35 may be of the direct conversion type for directly converting radiation 12 into electric signals with a plurality of pixels made of amorphous selenium (a-Se) or the like.

The radiation detector 36 converts radiation 12 that has passed through the subject 14 into radiographic image information, and supplies the radiographic image information as electric signals to the console 22, etc. As shown in FIG. 3, the radiation detecting device 18 includes, in addition to the circuit board 52 and the radiation detector 36, a battery 70, a cassette controller 72, and a transceiver 74, etc. The battery 70 serves as a power supply for the radiation detecting device 18. More specifically, electric power is supplied from the battery 70 to the radiation detector 36, the cassette controller 72, and the transceiver 74. The cassette controller 72 energizes the radiation detector 36 with electric power supplied from the battery 70. The transceiver 74 sends and receives signals, which represent information of the radiation 12 (radiographic image information) detected by the radiation detector 36, to and from the console 22, etc.

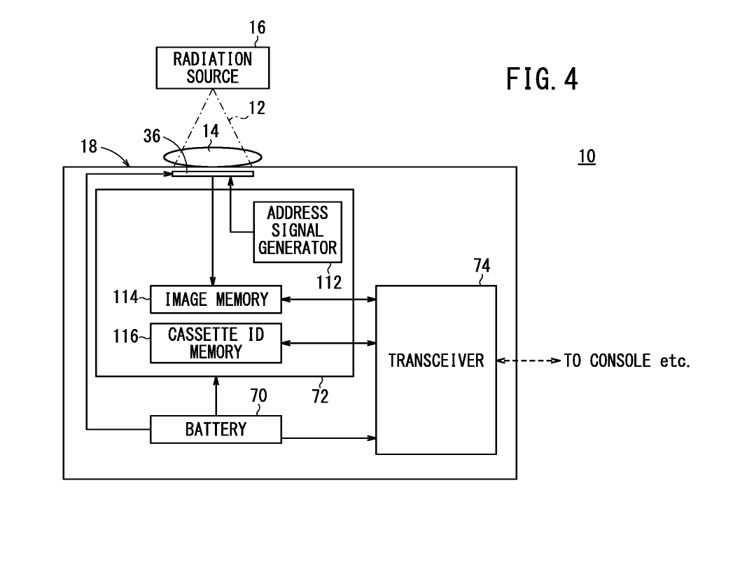

A circuit arrangement of the radiation detecting device 18 will be described in detail below with reference to FIGS. 3 and 4.

As shown in FIG. 3, the radiation detecting device 18 includes the photoelectric conversion layer 64 comprising pixels 62 made of a material such as a-Si or the like for converting visible light into electric signals. The photoelectric conversion layer 64 is disposed on the array of TFTs 60, which are arranged in rows and columns. The pixels 62 store electric charges generated by converting visible light into electric signals. The stored electric charges are read as image signals from the pixels 62 by successively turning on the rows of TFTs 60.

The TFTs 60, which are connected to the respective pixels 62, are connected to respective gate lines 94 that extend in parallel with the rows, and to respective signal lines 96 that extend in parallel with the columns. The gate lines 94 are connected to a line scanning driver 98, and the signal lines 96 are connected to a multiplexer 100. The gate lines 94 are supplied with control signals Von, Voff from the line scanning driver 98 for turning on and off the TFTs 60 along the rows. The line scanning driver 98 comprises a plurality of switches SW1 for switching between the gate lines 94, and a first address decoder 102 for outputting a selection signal for selecting one of the switches SW1 at a time. The first address decoder 102 is supplied with an address signal from the cassette controller 72.

The signal lines 96 are supplied with electric charges stored in the pixels 62 through the TFTs 60 arranged in the columns. The electric charges are amplified by amplifiers 104, which are connected to the multiplexer 100 through respective sample and hold circuits 106. The multiplexer 100 comprises a plurality of switches SW2 for successively switching between the signal lines 96, and a second address decoder 108 for outputting a selection signal for selecting one of the switches SW2 at a time. The second address decoder 108 is supplied with an address signal from the cassette controller 72. The multiplexer 100 is connected to an A/D converter 110. Radiographic image information, which is converted into digital signals by the A/D converter 110, is supplied to the cassette controller 72.

As shown in FIG. 3, the line scanning driver 98, the multiplexer 100, the amplifiers 104, the sample and hold circuits 106, and the A/D converter 110 are included in the electronic components 50 (see FIG. 2). Portions of the gate lines 94, which extend from the line scanning driver 98 to the photoelectric conversion layer 64, and portions of the signal lines 96, which extend from the photoelectric conversion layer 64 to the amplifiers 104, are included in the photoelectric transducer board 56 (see FIG. 2).

The TFTs 60, which function as switching elements, may be combined with any of various other image capturing devices such as a CMOS (Complementary Metal-Oxide Semiconductor) image sensor, or may be replaced with a CCD (Charge-Coupled Device) image sensor, in which electric charges are shifted and transferred by shift pulses that correspond to gate signals used in the TFTs.

As shown in FIG. 4, the cassette controller 72 of the radiation detecting device 18 includes an address signal generator 112, an image memory 114, and a cassette ID memory 116.

The address signal generator 112 supplies address signals to the first address decoder 102 of the line scanning driver 98, as well as to the second address decoder 108 of the multiplexer 100 shown in FIG. 3. The image memory 114 stores radiographic image information detected by the radiation detector 36. The cassette ID memory 116 stores cassette ID information for identifying the radiation detecting device 18.

The transceiver 74 sends the cassette ID information, which is stored in the cassette ID memory 116, and the radiographic image information, which is stored in the image memory 114, to the console 22, etc.

The casing 32 of the radiation detecting device 18 includes a cassette display unit 120 (see FIGS. 1, 6A, and 6B) for displaying messages indicating a sequence of radiographic image capturing processes and illustrations, to be described later.

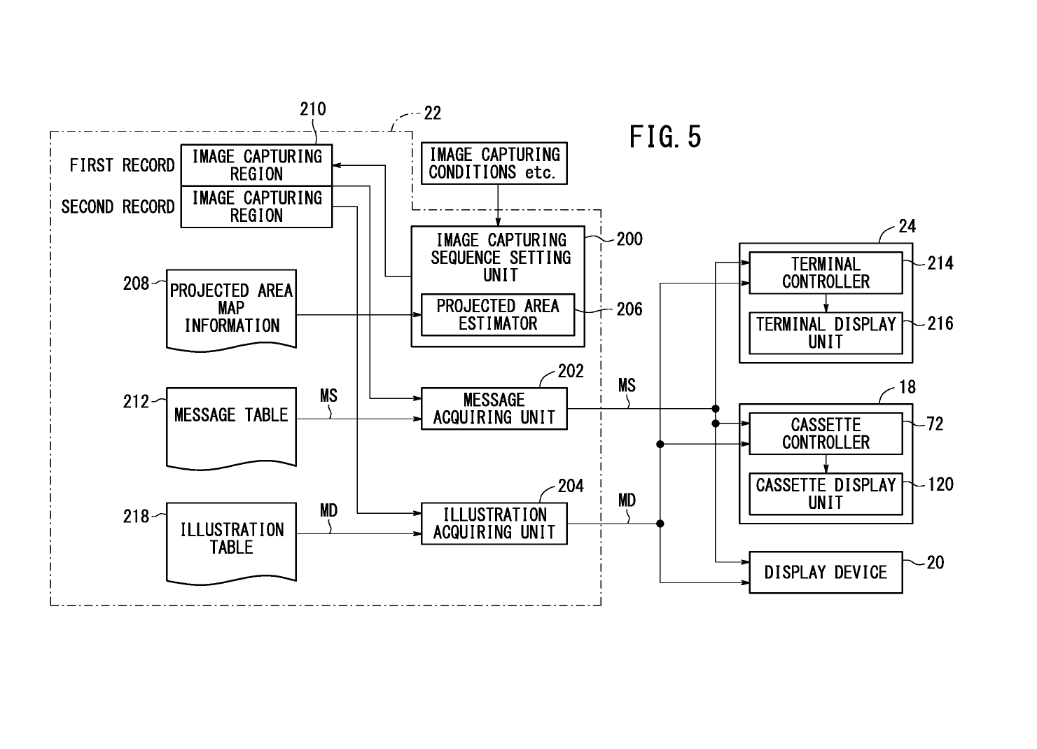

As shown in FIG. 5, the console 22 includes an image capturing sequence setting unit 200 for setting a radiographic image capturing sequence for carrying out a series of radiographic image capturing processes, a message acquiring unit 202 for acquiring a message representing the radiographic image capturing sequence and supplying the acquired message to the radiation detecting device 18, etc., and an illustration acquiring unit 204 for acquiring an illustration representing the radiographic image capturing sequence and supplying the acquired illustration to the radiation detecting device 18, etc. The series of radiographic image capturing processes represents radiographic image capturing processes that range from frontal to lateral radiographic image capturing processes, or from lateral to frontal radiographic image capturing processes, and basically refers to two or more radiographic image capturing processes according to a radiographic image capturing sequence designated by one image capturing order. The series of radiographic image capturing processes does not include radiographic image capturing processes that cover two persons.

The image capturing sequence setting unit 200 includes a projected area estimator 206 for estimating the projected area of an image capturing region, which is projected onto the radiation detecting device 18 prior to the series of radiographic image capturing processes. The image capturing sequence setting unit 200 sets the radiographic image capturing sequence such that the projected area in a first radiographic image capturing process will be equal to or greater than the projected area in a second radiographic image capturing process.

The projected area is estimated in the following manner. First, a reference projected area of each image capturing region is determined in advance, according to a computer graphics process, for example. For example, a standard outer profile (three-dimensional image information) for the subject 14 is established in advance. Using the irradiation surface 32a of the radiation detecting device 18 as a screen, and using the radiation source 16 as a camera viewpoint in the computer graphics process, while the distance between the irradiation surface 32a and the camera viewpoint is kept constant, perspective transformation is performed in order to determine screen coordinates of each of the image capturing regions. Then, a reference projected area at a constant distance from the determined screen coordinates is determined, and the reference projected area of each of the image capturing regions is stored as projected area map information 208 in a memory (not shown) of the console 22.

Prior to performing the series of radiographic image capturing processes, the projected area estimator 206 reads a reference projected area, which corresponds to each image capturing region, from the projected area map information 208, based on information of a plurality of image capturing regions included in the image capturing conditions, and corrects the reference projected area based on SID (Source-Image Distance) information of each of the image capturing regions included in the image capturing conditions, thereby estimating projected areas for the respective image capturing regions.

A preferred sequence of radiographic image capturing processes will be described below, based on the assumption that one radiographic image capturing process is carried out on a lateral chest region as an image capturing region (see FIG. 6A), and another radiographic image capturing process is carried out on a frontal chest region as an image capturing region (see FIG. 6B).

Capturing of radiographic images is carried out on image capturing regions, which are positioned centrally on the irradiation surface 32a of the radiation detecting device 18. Within the irradiation surface 32a of the radiation detecting device 18, a first region Za, which is irradiated with radiation 12 that has passed through the subject 14, is of a shape similar to and greater than the outer profile of the subject 14, and a second region Zb, which is irradiated with radiation 12 that has not passed through the subject 14, is of a frame shape surrounding the first region Za, with an outer contour similar to and greater than the outer contour of the subject 14. If the subject 14 is of significant thickness along the direction from the radiation detecting device 18 toward the radiation source 16, such as in a case where a radiographic image of a lateral chest region of the subject 14 is captured, then the tube voltage of the radiation source 16 should be set sufficiently high so that radiation 12 can pass through the subject 14. In this case, therefore, afterimage phenomena are highly likely to occur in the second region Zb. Such afterimage phenomena may refer to bright-burn phenomena if the radiation detecting device is of an indirect conversion type with a scintillator of CsI (cesium iodide), for example, or may refer to ghost phenomena if the radiation detecting device is of a direct conversion type made of selenium (Se), for example.

If a radiographic image of a lateral chest region of the subject 14 is captured as shown in FIG. 6A, and thereafter, a radiographic image of a frontal chest region of the subject 14 is captured as shown in FIG. 6B, then since the projected area of the lateral chest region that is projected onto the radiation detecting device 18 is smaller than the projected area of the frontal chest region that is projected onto the radiation detecting device 18, the second region Zb, which is irradiated during capturing of the radiographic image of the lateral chest region of the subject 14 (the region irradiated with radiation 12 that has not passed through the subject 14, see FIG. 6A), and the first region Za, which is irradiated during capturing of the radiographic image of the frontal chest region of the subject 14 (the region irradiated with radiation 12 that has passed through the subject 14, see FIG. 6B) overlap with each other. Therefore, the radiographic image, which is captured of the frontal chest region of the subject 14, is affected by afterimage phenomena that occur during capturing of the radiographic image of the lateral chest region of the subject 14.

Conversely, if a radiographic image of a frontal chest region of the subject 14 is captured, and thereafter, a radiographic image of a lateral chest region of the subject 14 is captured, then since the second region Zb, which is irradiated during capturing of the radiographic image of the frontal chest region of the subject 14, and the first region Za, which is irradiated during capturing of the radiographic image of the lateral chest region of the subject 14, do not overlap with each other, the radiographic image, which is captured of the lateral chest region of the subject 14, is not affected by afterimage phenomena that occur during capturing of the radiographic image of the frontal chest region of the subject 14.

As described above, the projected area estimator 206 of the image capturing sequence setting unit 200 estimates projected areas, which are projected onto the irradiation surface 32a of the radiation detecting device 18, of a plurality of image capturing regions defined in the image capturing conditions, based on the SID and information concerning the image capturing regions and the projected area map information 208. The image capturing sequence setting unit 200 sets a sequence of radiographic image capturing processes, such that the projected area of a first radiographic image capturing process will be equal to or greater than the projected area of a second radiographic image capturing process. Then, as shown in FIG. 5, the image capturing sequence setting unit 200 stores information of an image capturing region, which is set as an image capturing region to be imaged in the first radiographic image capturing process, in a first record of a sequence table 210, and stores information of an image capturing region, which is set as the image capturing region to be imaged in the second radiographic image capturing process, in a second record of the sequence table 210. If the tube voltage of the radiation source 16 is taken into account, then the image capturing sequence setting unit 200 sets the sequence of radiographic image capturing processes such that the projected area of the first radiographic image capturing process will be equal to or greater than the projected area of the second radiographic image capturing process, and so that the tube voltage in the first radiographic image capturing process is equal to or less than the tube voltage in the second radiographic image capturing process. Of course, the image capturing sequence setting unit 200 may set the sequence of radiographic image capturing processes while taking into account only the sizes of the projected areas, and not the magnitudes of the tube voltages.

The message acquiring unit 202 uses a message table 212 storing therein messages MS corresponding to respective image capturing regions, i.e., text data representing “frontal chest region”, “lateral chest region”, etc., as names of the image capturing regions. Prior to carrying out the first radiographic image capturing process, the message acquiring unit 202 reads from the message table 212 the message MS corresponding to the image capturing region stored in the first record of the sequence table 210, and supplies the read message MS to the radiation detecting device 18, the portable information terminal 24, and the display device 20. After the first radiographic image capturing process is completed, the message acquiring unit 202 reads from the message table 212 the message MS corresponding to the image capturing region stored in the second record of the sequence table 210, and supplies the read message MS to the radiation detecting device 18, the portable information terminal 24, and the display device 20.

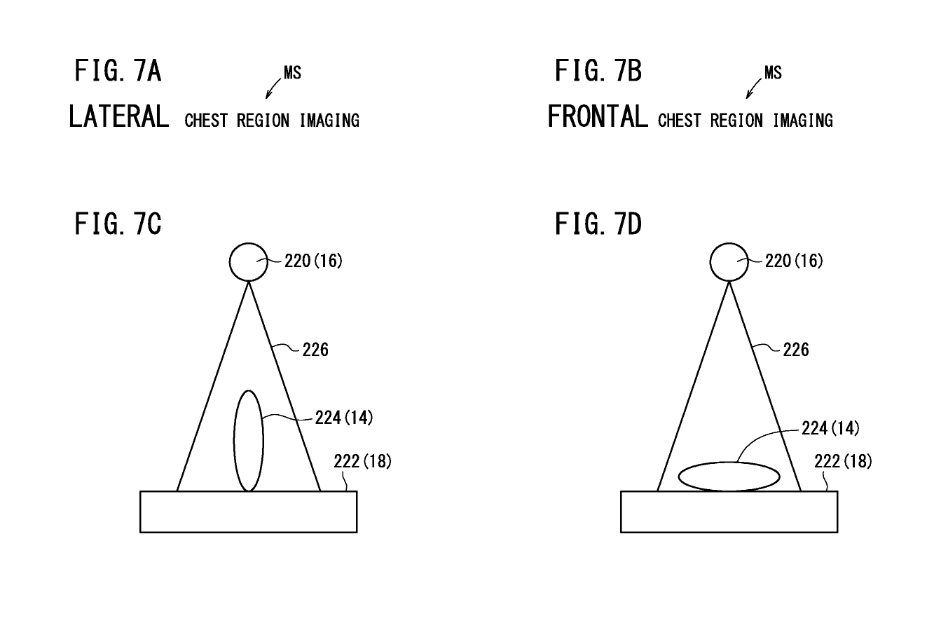

The cassette controller 72 of the radiation detecting device 18 receives a message MS from the message acquiring unit 202, and supplies the received message MS to the cassette display unit 120. The cassette display unit 120 displays the received message MS. Similarly, a terminal controller 214 of the portable information terminal 24 receives the message MS from the message acquiring unit 202, and supplies the received message MS to a terminal display unit 216, whereupon the terminal display unit 216 displays the received message MS. The display device 20 also receives the message MS from the message acquiring unit 202, and displays the received message MS on a display screen thereof. By way of example, FIG. 7 shows a message MS, which indicates capturing of an image of the lateral chest region, whereas FIG. 7B shows a message MS, which indicates capturing of an image of the frontal chest region.

The illustration acquiring unit 204 utilizes an illustration table 218, in which there are stored illustration data MD corresponding to each image capturing region, i.e., simplified image data representing “frontal chest region”, “lateral chest region”, etc. Prior to carrying out the first radiographic image capturing process, the illustration acquiring unit 204 reads from the illustration table 218 the illustration data MD, which corresponds to the image capturing region stored in the first record of the sequence table 210, and supplies the read illustration data MD to the radiation detecting device 18, the portable information terminal 24, and the display device 20. After completion of the first radiographic image capturing process, the illustration acquiring unit 204 reads from the illustration table 218 the illustration data MD, which corresponds to the image capturing region stored in the second record of the sequence table 210, and supplies the read illustration data MD to the radiation detecting device 18, the portable information terminal 24, and the display device 20.

The cassette controller 72 of the radiation detecting device 18 receives illustration data MD from the illustration acquiring unit 204, and supplies the received illustration data MD to the cassette display unit 120, whereupon the cassette display unit 120 displays the received illustration data MD. Similarly, the terminal controller 214 of the portable information terminal 24 receives illustration data MD from the illustration acquiring unit 204, and supplies the received illustration data MD to the terminal display unit 216, whereupon the terminal display unit 216 displays the received illustration data MD. The display device 20 also receives the illustration data MD from the message acquiring unit 202, and displays the received illustration data MD on the display screen thereof.

As shown in FIGS. 7C and 7D, an illustration may be an image represented by a FIG. 220 (circular) symbolizing the radiation source 16, a FIG. 222 symbolizing the radiation detecting device 18, and a FIG. 224 symbolizing the subject 14, such that the positional relationship between the FIGS. 220, 222, 224 is shown. For example, if the image capturing region is a lateral chest region, then as shown in FIG. 7C, the illustration may be an image represented by the FIG. 220, which symbolizes the radiation source 16, and the FIG. 224, which symbolizes the subject 14, e.g., the chest, one side of which is held in contact with one side of the FIG. 222 that represents the irradiation surface 32a. On the other hand, if the image capturing region is a frontal chest region, then as shown in FIG. 7D, the illustration may be an image represented by the FIG. 220, which symbolizes the radiation source 16, and the FIG. 224, which symbolizes the subject 14, e.g., the chest, the front of which is held in contact with one side of the FIG. 222 that represents the irradiation surface 32a. Each of the illustrations may further include the FIG. 226 that symbolizes the radiation 12 from the radiation source 16. Normally, the displayed messages MS are sufficient. However, the illustrations, which are displayed in addition, assist the operator in determining at a glance which image capturing region is to be imaged, and prevent the operator from making mistakes in relation to understanding the sequence of radiographic image capturing processes. In particular, the additional displayed illustrations are effective if the operator is a foreign operator who is unable to read messages that may be written in another language, such as Japanese, for example.

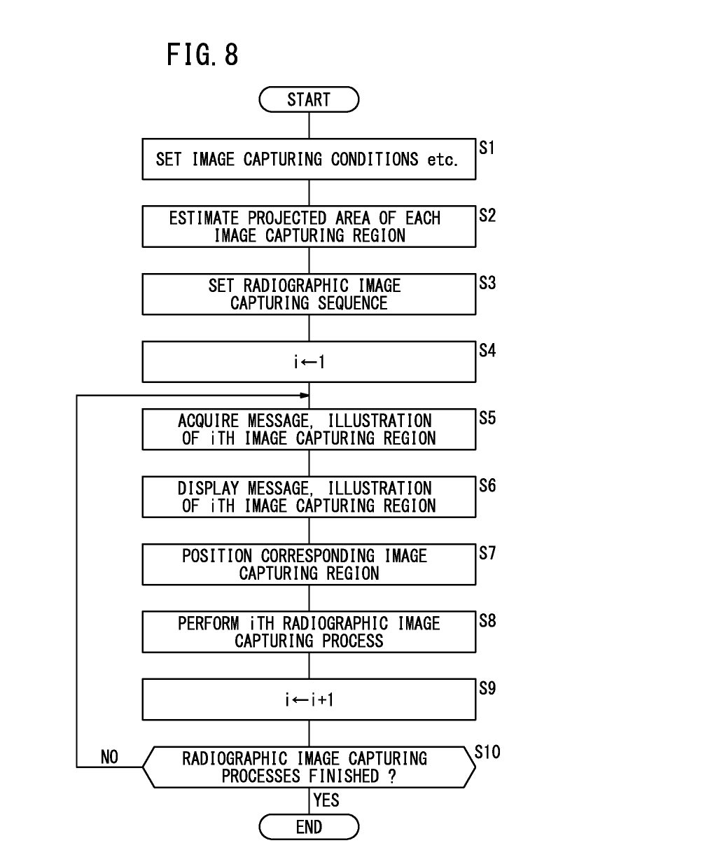

The radiographic image capturing system 10 according to the present embodiment is constructed basically as described above. Next, operations of the radiographic image capturing system 10 will be described below with reference to FIG. 8.

In step S1 of FIG. 8, the operator sets information of the subject 14, the image capturing conditions, etc., using the console 22. The image capturing conditions include information concerning a plurality of image capturing regions, tube voltages for the respective image capturing regions, etc., for example. The information of the subject 14, the image capturing conditions, etc., which have been set, are sent to the portable information terminal 24 in the possession of the operator, and are displayed on the terminal display unit 216 (see FIG. 5) of the portable information terminal 24. Accordingly, the operator can confirm the information of the subject 14, the image capturing conditions, etc., that are displayed on the terminal display unit 216, and can undertake desired preparations in order to capture radiographic images.

In step S2, the projected area estimator 206 estimates projected areas for the respective image capturing regions, which are projected onto the irradiation surface 32a of the radiation detecting device 18, based on information of the image capturing regions, the SID, and the projected area map information 208 of the image capturing conditions. The estimating process has already been described above, and thus will not be described in detail below.

In step S3, the image capturing sequence setting unit 200 sets a radiographic image capturing sequence based on the estimated projected areas for the respective image capturing regions. For example, if two radiographic image capturing processes are to be carried out, then the image capturing sequence setting unit 200 sets a radiographic image capturing sequence, such that the projected area of the first radiographic image capturing process will be equal to or greater than the projected area of the second radiographic image capturing process. Then, the image capturing sequence setting unit 200 stores information of an image capturing region, which has been set as the image capturing region to be imaged in the first radiographic image capturing process, in the first record of the sequence table 210, and stores information of an image capturing region, which has been set as the image capturing region to be imaged in the second radiographic image capturing process, in the second record of the sequence table 210. For example, if the information set in step S2 indicates that radiographic images of a frontal chest region and a lateral chest region are to be captured, then in step S3, information concerning the frontal chest region is stored in the first record of the sequence table 210, and information concerning the lateral chest region is stored in the second record of the sequence table 210.

In step S4, the value of a counter i for counting the number of radiographic image capturing processes is set to an initial value of “1”.

In step S5, the message acquiring unit 202 and the illustration acquiring unit 204 acquire a message MS and illustration data MD, respectively, which indicate that an image capturing region is to be imaged in a radiographic image capturing process represented by the value of the counter i (hereinafter referred to as an “ith radiographic image capturing process”), and the message MS and the illustration data MD are supplied, respectively, to the radiation detecting device 18, the portable information terminal 24, and the display device 20. More specifically, the message acquiring unit 202 reads a message MS corresponding to an image capturing region, i.e., a message indicating that the image capturing region is to be imaged, which is stored in a record of the sequence table 210 represented by the value of the counter i (hereinafter referred to as an “ith record”), from the message table 212, and supplies the read message MS to the radiation detecting device 18, the portable information terminal 24, and the display device 20. The illustration acquiring unit 204 reads illustration data MD corresponding to the image capturing region, i.e., illustration data indicating that the image capturing region is to be imaged, which is stored in the ith record of the sequence table 210, and supplies the read illustration data MD to the radiation detecting device 18, the portable information terminal 24, and the display device 20.

In step S6, the cassette display unit 120 of the radiation detecting device 18, the terminal display unit 216 of the portable information terminal 24, and the display device 20 display the message MS and the illustration data MD, which have been supplied thereto. Alternatively, the cassette display unit 120, the terminal display unit 216, and the display device 20 may display only one of the message MS and the illustration data MD.

In step S7, the operator confirms the message MS and the illustration displayed on the cassette display unit 120 of the radiation detecting device 18, the terminal display unit 216 of the portable information terminal 24, or the display device 20, and positions the subject 14 with respect to the radiation detecting device 18, so that a radiographic image of the image capturing region indicated by the message MS and the illustration can be captured.

In step S8, in response to the operator turning on an image capturing switch, the console 22 controls the radiation source 16 and the radiation detecting device 18, etc., in order to carry out the ith radiographic image capturing process.

In step S9, the value of the counter i is incremented by +1.

In step S10, it is judged whether or not all the radiographic image capturing processes have been completed, based on whether the value of the counter i is greater than the number of radiographic image capturing processes set in the image capturing conditions.

If all of the radiographic image capturing processes have not been completed, the process from step S5 is repeated. If all of the radiographic image capturing processes have been completed, the operation sequence of the radiographic image capturing system 10 is brought to an end.

If the information set in step S1 indicates that radiographic images of a frontal chest region and a lateral chest region are to be captured, then in step S6, prior to carrying out the first radiographic image capturing process, the message MS and the illustration, which indicate that the frontal chest region is to be imaged, are displayed on the display screens of the cassette display unit 120, the terminal display unit 216, and the display device 20. Therefore, in step S7, the operator positions the subject 14 by placing the frontal chest region against the irradiation surface 32a of the radiation detecting device 18, and then in step S8, the operator carries out the first radiographic image capturing process. Thereafter, in step S6, prior to carrying out the second radiographic image capturing process, the message MS and the illustration, which indicate that the lateral chest region is to be imaged, are displayed on the display screens of the cassette display unit 120, the terminal display unit 216, and the display device 20. Therefore, in step S7, the operator positions the subject 14 by placing the lateral chest region against the irradiation surface 32a of the radiation detecting device 18, and then in step S8, the operator carries out the second radiographic image capturing process. Therefore, it is possible for the second radiographic image capturing process to be performed without being affected by afterimage phenomena that may have occurred in a case where the first radiographic image capturing process was performed.

The radiographic image capturing system 10 according to the present embodiment includes the image capturing sequence setting unit 200 for setting the radiographic image capturing sequence, and the image capturing sequence display unit (the cassette display unit 120, the terminal display unit 216, and the display device 20) for displaying the set radiographic image capturing sequence. Consequently, before a series of radiographic image capturing processes is carried out, it is possible to set the radiographic image capturing sequence for capturing radiographic images that stay clear of regions in which afterimage phenomena tend to occur, and to display the set radiographic image capturing sequence. The operator can capture radiographic images that exclude regions in which afterimage phenomena occur by performing radiographic image capturing processes according to the displayed radiographic image capturing sequence. As a result, the S/N ratio and contrast are prevented from being lowered, and a reduction in cost can be achieved without lowering the degree of freedom in designing the radiation detecting device 18.

Before radiographic image capturing processes are carried out, the projected area estimator 206 of the image capturing sequence setting unit 200 estimates the projected area of an image capturing region, which is projected onto the radiation detecting device 18, and the image capturing sequence setting unit 200 sets the radiographic image capturing sequence such that the projected area of the first radiographic image capturing process will be equal to or greater than the projected area of the second radiographic image capturing process. Consequently, it is possible to set a radiographic image capturing sequence in order to capture radiographic images that reliably exclude regions where afterimage phenomena tend to occur. Further, in view of the tube voltage of the radiation detecting device 18, the image capturing sequence setting unit 200 may set a radiographic image capturing sequence such that the projected area of the first radiographic image capturing process will be equal to or greater than the projected area of the second radiographic image capturing process, and such that the tube voltage in the first radiographic image capturing process is equal to or less than the tube voltage in the second radiographic image capturing process. In this manner, it is possible to set a radiographic image capturing sequence in order to capture radiographic images that more reliably exclude regions where afterimage phenomena tend to occur.

In the illustrated embodiment, two radiographic image capturing processes are carried out. However, the present invention may also be applied to a sequence of three or more radiographic image capturing processes. In this case, the radiographic image capturing sequence may be set such that the projected area of the first radiographic image capturing process is the greatest, and the projected area becomes progressively smaller as the number of radiographic image capturing processes is greater.

The radiographic image capturing system and the radiographic image capturing method according to the present invention are not limited to the embodiment described above, but various alternative or additional configurations may be adopted therein without departing from the scope of the present invention.

For example, image capturing conditions, which are changed for different image capturing regions, may be defined in the following manner.

(a) Only the tube voltage is changed for each image capturing region.

(b) Only the tube current is changed for each image capturing region.

(c) Only time is changed for each image capturing region.

(d) Two of the tube voltage, the tube current, and time are changed for each image capturing region.

(e) All of the tube voltage, the tube current, and time are changed for each image capturing region.

Furthermore, radiographic image capturing processes may be carried out under image capturing conditions that are changed for one image capturing region. Such radiographic image capturing processes may be carried out under the aforementioned image capturing conditions (a) through (e).

For example, not only radiographic images of respective frontal and lateral chest regions, but also a plurality of radiographic images of only one frontal chest region may be captured under different image capturing conditions.

If the subject is slightly displaced at the time that the radiographic image capturing processes are carried out under different image capturing conditions, then radiation that has passed through the subject may possibly be applied to a region where afterimage phenomena has occurred (a region irradiated with radiation that has not passed through the subject is established originally on the assumption that the subject may be displaced). Therefore, it is preferable to carry out the radiographic image capturing processes according to a sequence for minimizing the effect of afterimage phenomena. Accordingly, the image capturing sequence setting unit 200 sets a sequence of radiographic image capturing processes such that at least one of the tube voltage, the tube current, and time will change from a smaller value to a greater value during the radiographic image capturing processes.

In this manner, radiographic image capturing processes can be carried out in succession, virtually without being affected by afterimage phenomena, in the same manner as in a case where the radiographic image capturing sequence is set based on projected areas. For example, since the tube voltage is higher, more radiation 12 reaches the radiation detecting device 18, thereby making the radiation detecting device 18 more susceptible to afterimage phenomenon. Therefore, the image capturing sequence setting unit 200 preferably sets the sequence of radiographic image capturing processes such that the tube voltage will change from a smaller value to a greater value during the radiographic image capturing processes.

1. A radiographic image capturing system comprising:
a radiation source;
a radiation detecting device including a casing and a radiation detector housed in the casing for converting radiation emitted from the radiation source and that has passed through at least a subject into radiographic image information;
an image capturing sequence setting unit for setting a radiographic image capturing sequence for carrying out a series of radiographic image capturing processes; and
an image capturing sequence display unit for displaying the set radiographic image capturing sequence.

2. The radiographic image capturing system according to claim 1, wherein the image capturing sequence setting unit includes:
a projected area estimator for estimating a projected area of an image capturing region, which is projected onto the radiation detecting device prior to the series of radiographic image capturing processes; and
the image capturing sequence setting unit sets the radiographic image capturing sequence such that the projected area will change from a greater value to a smaller value during the radiographic image capturing processes.

3. The radiographic image capturing system according to claim 1, wherein the image capturing sequence setting unit sets the radiographic image capturing sequence such that at least one of a tube voltage, a tube current, and time will change from a smaller value to a greater value during the radiographic image capturing processes.

4. The radiographic image capturing system according to claim 1, wherein the image capturing sequence setting unit sets the radiographic image capturing sequence such that a tube voltage will change from a smaller value to a greater value during the radiographic image capturing processes.

5. The radiographic image capturing system according to claim 1, wherein the image capturing sequence display unit is connected to a controller, which controls at least the radiation source and the radiation detecting device.

6. The radiographic image capturing system according to claim 1, wherein the image capturing sequence display unit is mounted on the casing of the radiation detecting device.

7. The radiographic image capturing system according to claim 1, wherein the image capturing sequence display unit is mounted on a portable information terminal, which is carried by an operator of the radiographic image capturing system.

8. The radiographic image capturing system according to claim 1, wherein the radiation detecting device comprises:
a scintillator using CsI (cesium iodide) for temporarily converting the radiation into visible light; and
solid-state detecting elements for converting the visible light into electric signals.

9. A radiographic image capturing method using:
a radiation source; and
a radiation detecting device including a casing and a radiation detector housed in the casing for converting radiation emitted from the radiation source and that has passed through at least a subject into radiographic image information;
the radiographic image capturing method comprising:
an image capturing sequence setting step of setting a radiographic image capturing sequence for carrying out a series of radiographic image capturing processes; and
an image capturing sequence display step of displaying the set radiographic image capturing sequence.

10. The radiographic image capturing method according to claim 9, wherein the image capturing sequence setting step further comprises a projected area estimating step of estimating a projected area of an image capturing region, which is projected onto the radiation detecting device prior to the series of radiographic image capturing processes; and
the image capturing sequence setting step sets the radiographic image capturing sequence such that the projected area will change from a greater value to a smaller value during the radiographic image capturing processes.

11. The radiographic image capturing method according to claim 9, wherein the image capturing sequence setting step sets the radiographic image capturing sequence such that at least one of a tube voltage, a tube current, and time will change from a smaller value to a greater value during the radiographic image capturing processes.

12. The radiographic image capturing method according to claim 9, wherein the radiation detecting device comprises:
a scintillator using CsI (cesium iodide) for temporarily converting the radiation into visible light; and
solid-state detecting elements for converting the visible light into electric signals.
